(19) United States
(12) Patent Application Publication
(10) Pub. No.: US 2019/0241863 A1
(43) Pub. Date: Aug. 8, 2019

(54) ENHANCED BREAD-MAKING YEASTS

(71) Applicant: LESAFFRE ET COMPAGNIE, Paris (FR)

(72) Inventors: Jean-Charles BARTOLUCCI, Hellemmes-Lille (FR); Evelyne FONCHY-PENOT, Fleurbaix (FR); Jennifer IMBERT-PODGORSKI, Mons en Baroeul (FR); Florence KAPRAL, Comines (FR); Georges PARASIE, Sequedin (FR); Anne-Dominique QUIPOURT-ISNARD, Marc en Baroeul (FR); Valérie TRIONE, Comines (FR)

(73) Assignee: LESAFFRE ET COMPAGNIE, Paris (FR)

(21) Appl. No.: 16/318,731

(22) PCT Filed: Jul. 19, 2017
(86) PCT No.: PCT/FR2017/051970

§ 371 Date: Jan. 18, 2019

(30) Foreign Application Priority Data
Jul. 20, 2016 (FR) 1656902

(51) Int. Cl. C12N 1/18; C12R 1/865; A21D 8/04

(52) U.S. Cl. CPC C12N 1/18; A21D 8/047; C12R 1/865

(57) ABSTRACT

The present invention relates to bread-making yeast strains, also known as baker's yeasts. In particular, the present invention relates to the yeast strain deposited on May 19, 2016, with the CNCM under No. I-5088. This strain is particularly advantageous for use in a bread-making process, particularly a bread-making process of “indirect” type, also known as “Sponge and Dough”.

### TECHNICAL FIELD OF THE INVENTION

The present invention relates to bread-making yeast strains, also known as baker's yeasts. Such strains are of particular interest in the production of bread products. The present invention relates particularly to a yeast strain deposited on May 19, 2016, with the CNCM [French National Collection of Microorganism Cultures] under No. 1-5088.

### TECHNOLOGICAL BACKGROUND

The production of bread-making products on an industrial scale requires the use of “baker's” yeasts. Such yeasts must allow efficient large-scale fermentation for various bread-making processes.

The process of “Sponge and Dough” type is a bread-making process very widely used in the industrial sector. During this process, the breadmaking is carried out with two fermentation steps: a first “SPONGE” step which corresponds to a first fermentation of a dough comprising more than half the total flour used, a portion of the water and all or part of the yeast, and a second “DOUGH” fermentation step, in which the SPONGE is combined with the rest of the flour, the rest of the water and the other ingredients of the dough. Such a bread-making process requires the use of strains which have high fermentation capacities making it possible in particular to obtain a strong release of gas (CO₂) particularly during the Dough phase.

At the current time, the reference strain used for this type of fermentation is the strain corresponding to the yeast sold under the reference Red Star Cream Yeast, hereinafter denoted RSCY, which has good fermentation capacities and which is compatible with industrial use. Nevertheless, in an industrial use scheme, it would be very advantageous to:
develop yeasts which have even higher fermentation capacities; and/or
reduce the amount of yeasts used;
   and/or
reduce the time required for the fermentation of the dough.

### DETAILED DESCRIPTION OF THE INVENTION

In this context, the present inventors have developed several yeast strains which have improved fermentation capacities compared with the RSCY reference strain.

The yeasts developed in the context of the present invention have been obtained by mutagenesis of the RSCY reference strain, and selected because they make it possible to increase the amount of gas given off by approximately 10% in the Dough compared with the RSCY reference strain, and/or to reduce by 10% the amount of yeast solids used and/or to reduce by 10% the time required for the fermentation.

The inventors have particularly developed the yeast strain deposited on May 19, 2016, at the CNCM (Collection Nationale de Cultures de Micro-organismes, 25/28 rue du docteur ROUX, F-75724 Paris Cedex 15) pursuant to the treaty of Budapest under No. 1-5088. This strain has a fermentative capacity on a Sponge and Dough scheme that is much higher than that recorded with the reference strain, with in particular an increase in amount of gas given off of 9-10% compared to the RSCY strain, this being in a 5 to 12% shorter period of time.

Thus, in a first aspect, the present invention relates to a yeast strain which is the strain deposited on May 19, 2016, at the CNCM pursuant to the treaty of Budapest under number 1-5088.

The expression “yeast strain” denotes a relatively homogeneous population of yeast cells. A yeast strain is obtained from a clone, a clone being a population of cells that is obtained from a single yeast cell.

The yeast strains thus modified are particularly advantageous since they can be used in an industrial-scale bread-making process.

The present invention also relates to a yeast from a yeast strain as described above.

The yeast strain according to the present invention was obtained by means of a process comprising a step of mutagenesis of the RSCY reference strain, followed by a step of selection of the mutants thus obtained according to their fermentative capacities.

The mutagenesis consists in introducing mutations into the genetic material of the yeast by exposing the latter to mutagenic agents. An example of a mutagenic agent that may be used in the context of the present invention is UV radiation.

The mutagenesis program gave rise to the production of a very large number of mutants, virtually 10 000 mutants. Said mutants integrated a complex selection process comprising several levels of evaluation, always based on the comparison of the ferment power of these mutants compared with the reference strain.

Finally, the mutants having the best CO₂ release in Sponge and Dough but also in an application in a direct bread-making scheme, were retained.

The yeasts used for the breadmaking may be in several forms. For example, the yeast according to the invention may be in the form of cream yeast, compressed yeast, dry yeast or frozen yeast.

Fresh yeasts are characterized by a high water content compared with dry yeasts. Fresh yeasts encompass cream yeasts and compressed yeasts.

Cream yeasts, also known as “liquid yeasts”, are aqueous suspensions of yeast cells having a cream-type viscosity. These aqueous suspensions of living yeast cells generally have a solids contents of at least 12% by weight, particularly from 12 to 40% by weight. A cream yeast may have, for example, a solids content of between 12 and 25% by weight, preferably between 14 and 22% by weight.

Compressed yeasts comprise yeasts compressed into a compact block and also crumbled compressed yeasts. Compressed yeasts in a compact block, also known as “yeast bars”, are characterized by a solids content of between 26% and 35%. Crumbled compressed yeasts themselves have a water content of between 21% and 35%.

Dry yeasts are characterized by a solids content of greater than approximately 92%.

Frozen yeasts are characterized by a solid content of between 74% and 80%.

The invention also relates to the use of a yeast strain as described above, for producing bread-making products.

In one particular embodiment, the yeast according to the invention is in the form of cream yeast.

Breadmaking groups together all of the operations consisting in converting flour into bread.

The yeasts according to the invention may be used in bread-making processes of direct (“NO-TIME DOUGH”) scheme type or of indirect (“SPONGE and DOUGH”) scheme type.

A direct scheme comprises virtually no first fermentation between intensive kneading and the division of the dough, the dough pieces obtained being fermented in a mold between 35° C. and 40° C., then baked.

The yeasts according to the present invention are particularly effective in bread-making processes, in particular in a bread-making process of Sponge and Dough type. This type of process is described for example in the reference book “Bakers Handbook” by E. J. Pyler, published by Sosland Publishing Co.

Thus, according to another aspect, the present invention relates to the use of a yeast strain as described above, in a bread-making process of Sponge and Dough type.

A “SPONGE and DOUGH” scheme is a bread-making process comprising two fermentation steps:
a first step, called “SPONGE”, which corresponds to the fermentation of a dough comprising 50 to 70% of the total flour used, a portion of the water and all or part of the yeast, for several hours, in general between 3 and 6 hours, and more particularly between 3 and 4 hours; and
a second step, called “DOUGH”, in which the SPONGE obtained after the above fermentation is combined with the rest of the flour, the rest of the water and the other ingredients of the dough, the mixture thus formed is kneaded, divided, placed in a mold and fermented, then baked, this second in-mold fermentation corresponding to the proofing, its duration being the proof time.

The percentages are expressed as “baker's” percentages, the “baker's” percentage being a method of calculation applied to the ratios of ingredients, in which the total weight of the flour always represents 100% and the weight of the other ingredients of the dough is calculated relative to this weight of flour.

The present invention thus relates to a bread-making process of Sponge and Dough type comprising a step of fermentation by yeast according to the invention.

According to one particular embodiment, the invention pertains to a bread-making process, particularly of Sponge and Dough type, comprising a step of fermentation by a yeast according to the invention, characterized in that the proof time of the baking dough is less than 8%, particularly 10%, even more particularly 12%, compared with the time required for the fermentation of said baking dough by the RSCY reference strain.

According to another particular embodiment, the invention is pertains to a bread-making process, particularly of Sponge and Dough type, comprising a step of fermentation by a yeast according to the invention, characterized in that the amount of yeast required for the fermentation of the baking dough is less than 10% compared with the amount of yeast required for the fermentation of said baking dough with the RSCY reference strain.

The invention is also directed toward a baking dough comprising a yeast according to the invention. Particularly, the invention is directed toward a baking dough comprising yeasts of which at least 10% are yeasts as described above. In one particular embodiment, the baking dough according to the present invention comprises yeasts of which at least 25%, 50% or 75% are yeasts as described above.

The baking dough may be a non-sweetened dough, a slightly sweetened dough or a sweetened dough, the sugar usually being sugar syrup or sucrose.

The expression “slightly sweetened dough” denotes doughs which have a content of added sugar of less than 14% by weight relative to the weight of the flour, preferably less than or equal to 12% by weight relative to the weight of the flour.

The invention is also pertains to a process for preparing a baking dough, comprising a step of fermentation by a yeast according to the invention.

According to one particular embodiment, the process for preparing baking dough according to the present invention is of Sponge and Dough type.

### EXAMPLES

Obtaining Yeasts Having High Fermentative Capacities

A program of mutagenesis by UV bombardment was carried out on the RSCY reference strain, which gave rise to the production of numerous mutants. Said mutants integrated a complex selection process comprising several levels of evaluation, always based on the comparison of the ferment power of these strains compared with the RSCY reference strain. Thus, this process enabled the selection of the 1-5088 strain among the 10000 mutants evaluated.

The 1-5088 strain resulting from this selection process was produced in a 20 L fermenter and evaluated in Sponge and Dough breadmaking. Two test campaigns were carried out. For each test campaign, three tanks were prepared with the 1-5088 strain independently. The operation was carried out likewise with the RSCY reference strain.

The sponge and dough bread-making formula used is indicated in the following table. This formula corresponds to the situation where all the yeast is placed in the sponge. The amounts are expressed as baker's percentages, namely relative to the amount of flour used. The amount of yeast is adjusted depending on the solids content measured for each cream yeast, so as always to obtain an amount of 1.275% of yeast solids based on flour in the kneading machine. The amount of pouring water in the sponge is adjusted according to the amount of yeast, such that the total of the two is always equal to 42.5%.

| Ingredient | % in the sponge | % in the dough | total % |
|---|---|---|---|
| Flour | 70 | 30 | 100 |
| Water | 35.5 | 16 | 51.5 |
| Liquid yeast at 18.2% | 7 | — | 7 |
| Yeast food | 0.25 | — | 0.25 |
| Sodium stearoyl lactylate | 0.5 | — | 0.5 |
| Alpha-amylase | 0.0002 | — | 0.0002 |
| High Fructose | — | 15 | 15 |
| Corn Syrup | | | |
| Salt | — | 1.8 | 1.8 |
| Calcium propionate | — | 0.5 | 0.5 |
| Soybean oil | — | 3 | 3 |
| Monoglycerides | — | 1 | 1 |
| Vital Gluten | — | 1 | 1 |
| Lesoft improver | — | 0.25 | 0.25 |

The bread-making diagram followed was the following for preparing the sponge:
The jacket of the Hobart kneading machines is tempered with water at 22° C.
The pouring water used is at 24° C.+/−1° C.
The target dough temperature is 25° C.
The kneading takes place in Hobart kneading machines equipped with Mac Duffy tanks, according to the following diagram:
   The dry ingredients are premixed for 1 minute
   The liquid ingredients are then incorporated
   1 minute at speed 1
   2 minutes at speed 2
After the kneading, the doughs are removed from the tanks, and placed in mixing bowls which are placed in a chamber regulated at 30° C. and 90% RH for 4 hours. This is a bulk fermentation.

The bread-making diagram followed was the following for preparing the dough:
A further kneading is carried out as follows:
   The ingredients of the dough are placed in the kneading machine
   1 minute at speed 1
   Incorporation of the sponge
   1 minute at speed 1
   3 minutes at speed 2.
On exiting the kneading machine, the bulk fermentation time is 5 minutes, at the end of which the doughs are divided up (3 pieces of 516 g per kneading machine) and rolled into balls manually.
After slackening for 5 minutes, the dough pieces are shaped in a horizontal shaper and the shaped dough pieces are placed in molds which are placed in a chamber regulated at 43° C. and 90% relative humidity (RH).
At the end of this final fermentation phase, which is called proofing, baking is carried out for 23 minutes in a Reed tray oven, preheated to 180° C.
The breads are then removed from the oven and swept for 1 hour.

The three types of measurement are carried out in order to quantify the fermentative activity:
1. At the end of the kneading of the doughs, 50 g of each kneaded product are sampled and placed in a pot intended for the measurement of the gas production by a Risograph®. The gas accumulation after two hours (DG2H) is exploited.
2. At the time the molds are introduced into the chamber regulated at 43° C., templates are placed on each mold, the level of which exceeds the height of the mold by 1.2 cm. The time required for the dough present in the mold to reach this height is measured. This time is known as the Proof Time (PT), it is measured in minutes.
3. For certain dough pieces, baking is carried out after a constant time. The volume and the weight of the breads resulting from this baking are measured. The ratio of the volume to the weight makes it possible to estimate the specific volume (SV) of each bread, after a constant fermentation time.

The results obtained with the creams produced from the 1-5088 strain are related to those obtained with the RSCY reference strain. In the following table, the results are thus expressed as percentage difference.

| | mean % difference | | |
|---|---|---|---|
| | between three creams | | |
| | produced with I-5088 | | |
| | and three creams | | |
| | produced with RSCY | | |
| | % DG2H | % PT | % SV |
| Test campaign N^{o}1 | +9% | −5% | 0% |
| Test campaign N^{o}2 | +11% | −8% | +1% |

These results demonstrate that the 1-5088 strain makes it possible to obtain a gain in Proof Time of 5 to 8% and a gain in amount of gas released of 9-11% compared with the creams produced under the same conditions with the RSCY reference strain.

1. A yeast strain deposited on May 19, 2016, at the Collection Nationale de Cultures de Micro-organismes (CNCM) pursuant to the treaty of Budapest under No. 1-5088.

2. A yeast from the yeast strain as claimed in claim 1.

3. The yeast as claimed in claim 2, characterized in that it is in a cream yeast form.

4. A bread-making process, comprising a step of using a yeast as claimed in claim 2.

5. The bread-making process as claimed in claim 4, characterized in that said bread-making process is of Sponge and Dough type.

6. The bread-making process use as claimed in claim 5, characterized in that said process requires an amount of yeast of less than 10% compared with the amount of yeast required when the RSCY reference strain is used.

7. A baking dough obtained by the bread-making process as claimed in claim 4.
